(19) United States
(12) Patent Application Publication
(10) Pub. No.: US 2026/0327571 A1
(43) Pub. Date: Oct. 22, 2026

(54) METHOD FOR REPROGRAMMING SOMATIC CELLS TO GENERATE INDUCED PLURIPOTENT STEM CELLS

(71) Applicants: ICAMUNO BIOTHERAPEUTICS (SUZHOU) CO., LTD., Suzhou, Jiangsu (CN); ICAMUNO BIOTHERAPEUTICS (HANGZHOU)CO., LTD., Hangzhou, Zhejiang (CN)

(72) Inventor: Xiaodong LIU, Hangzhou, Zhejiang (CN)

(73) Assignees: ICAMUNO BIOTHERAPEUTICS (SUZHOU) CO., LTD., Suzhou, Jiangsu (CN); ICAMUNO BIOTHERAPEUTICS (HANGZHOU)CO., LTD., Hangzhou, Zhejiang (CN)

(21) Appl. No.: 19/484,885

(22) PCT Filed: May 15, 2024
(86) PCT No.: PCT/CN2024/093355

§ 371 Date: Nov. 17, 2025

(30) Foreign Application Priority Data
May 17, 2023 (CN) 202310558020.X

(51) Int. Cl. C12N 5/074; C12N 5/00; C12N 15/11; C12N 15/86

(52) U.S. Cl. CPC C12N 5/0696; C12N 5/0062; C12N 15/11; C12N 15/86; C12N 2501/602; C12N 2501/603; C12N 2501/604; C12N 2501/606; C12N 2501/727; C12N 2506/11; C12N 2506/1307; C12N 2510/00; C12N 2513/00; C12N 2760/18843; C12N 2800/10

(57) ABSTRACT

A method used to generate induced pluripotent stem cells by reprogramming somatic cells (particularly human cells and non-human primate somatic cells) in 3-dimensional (3D) suspension culture.

### FIELD

The present invention relates to a method for reprogramming somatic cells, in particular to a method for producing induced pluripotent stem cells by reprogramming somatic cells (particularly human somatic cells) in 3-dimension (3D) suspension culture.

### BACKGROUND

Human blastocyst-derived embryonic stem cells (ESCs) have demonstrated pluripotency. For instance, they have self-renew potential and can differentiate into all germ layer cells¹. Embryonic stem cells, due to their pluripotent property, are promising for generating various cell derivatives that are applicable in both research and clinical practice. Subsequent advances in this field found that terminally differentiated somatic cells can be induced into a pluripotent state similar to that of ESCs via nuclear reprogramming mediated by OKSM (OCT4, KLF4, SOX2, and c-MYC), resulting in induced pluripotent stem cells (iPSCs) ². The revolutionary discovery of human iPSCs avoids many ethical issues related to human ESC production. Human iPSCs are similar to human ESCs and therefore serves as a potential alternative in cell therapy and regenerative medical applications^{3, 4}.

To date, various reprogramming methods and advancements for generating iPSCs have been reported, aiming to improve the safety, stability, quality, and quantity of iPSCs to meet the current requirements for manufacture of iPSC derivatives in clinical. For example, in order to replace traditional reprogramming methods using integrating lentiviruses/retroviruses, reprogramming methods using non-integrating viruses/mRNA//episomal/protein have been developed to generate transgene-free iPSCs2,5-10. In addition, it has been reported that modifications to the reprogramming process, for instance, the addition of hydrogels to provide a three-dimensional microenvironment for reprogramming, can enhance the reprograming^{11,12}. The 3-dimensional (3D) culture system has been developed for culturing iPSCs, which uses large-scale bioreactors to culture spheroid-shaped iPSCs. The 3D culture system has demonstrated a comparable ability to maintain pluripotency and enable a larger scale production of iPSCs compared to the traditional 2-dimensional (2D) culture system (i.e., monolayer culture)¹³⁻¹⁶.

Although iPSC technology has been proven promising in the field of cell therapy and regenerative medicine, it is still a long process to produce a sufficient amount of iPSCs from somatic cell sources for therapeutic purposes in the present field. There is a need in the field for a reprogramming method to achieve a greater iPSC output in a shorter time period.

### SUMMARY

Conceptually, integrating the 3D reprogramming with the 3D culture system is a promising strategy to increase iPSC output. However, feasibility studies on this integration are very limited, with the current majority focusing on mouse fibroblasts. Integration of reprogramming with 3D culture systems on human somatic cells has not been reported to date. For example, referring to doi: 10.1038/nmeth. 1939 (Derivation, expansion and differentiation of induced pluripotent stem cells in continuous suspension cultures, 2016), published is the generation of induced pluripotent stem cells by culturing transfected mouse fibroblasts in suspension in the very early phase of the reprogramming process (approximately 2-3 days after transduction of the OKSM reprogramming factors). The authors pointed out that although the suspension culture can yield iPSC populations, they were non-uniform due to the absence of clonal selection and comprised partially reprogrammed cells that would impact the subsequent differentiation potential of such populations.

Furthermore, it has been pointed out by studies that although mouse and human cells can be reprogrammed by using the same combination of factors, the two have significant differences in reprogramming processes. The reprogramming of mouse cells can usually be finished within a week or two, whereas the reprogramming of human cells may take up to a month. The conversion into human iPSCs is significantly less efficient than that of mouse cells, and the human iPSCs have a different characteristic morphology from that of mouse iPSCs. In addition, studies have also found significant differences in the actual regulatory networks of pluripotency factors between the two species in the early phase of reprogramming. These differences make it difficult to extrapolate and apply findings of reprogramming studies in mouse cells to human cells (See, for example https://doi.org/10.1186/s12864-018-5326-1 (Comparison of reprogramming factor targets reveals both species-specific and conserved mechanisms in early iPSC reprogramming, 2018) and https://reactome.org/PathwayBrowser/#R-HSA-452723&FLG-043474, DOI: 10.3180/REACT 200812.1).

In order to address the aforementioned issues, the inventors of the present invention, through devoting themselves to studies on reprogramming of human somatic cells, surprisingly found that introducing 3D suspension culture into the relatively early stage of reprogramming in which mesenchymal-to-epithelial transition (MET) occurs is an effective strategy for integrating reprogramming with 3D culture on human cells. Based on this finding, the inventors of the present invention develop the 3D reprogramming method of the present invention. While not wishing to be bound by any theory, it is believed that integrating 3D suspension culture in the relatively early stage of reprogramming where MET occurring, will only allow stably reprogrammed cells to continue to proliferate and produce iPSC spheroids. As a result, the reprogramming method of the present invention does not require the conventional clonal selection to remove persistently unreprogrammed or unstably partially reprogrammed cells from the culture, thereby effectively simplifying the procedure. Furthermore, as shown in some embodiments of the present invention, the method of the present invention allows a larger scalable production of stable iPSCs with better differentiation potential within a shorter time window as compared to the conventional 2D reprogramming method.

Accordingly, in a first aspect, the present invention provides a method for producing an induced pluripotent stem cell (iPSC) from a somatic cell by reprogramming, wherein the somatic cell is a human or non-human primate somatic cell. The method comprises culturing a self-reprogramming intermediate in a 3D suspension to produce spheroids of reprogrammed iPSCs. In the present invention, the method is also referred to as the “3D reprogramming method of the present invention”. In the present invention, an iPSC produced according to the 3D reprogramming method of the present invention, is also referred to as a “3D-reprogrammed iPSC”.

In some embodiments, the 3D reprogramming method of the present invention comprises steps of:
(a) contacting a somatic cell with a reprogramming factor to perform reprogramming treatment on the somatic cell,
(b) culturing the somatic cell treated in step (a) under a 2D culture condition for a period sufficient to obtain a reprogramming intermediate, wherein the reprogramming intermediate is characterized by at least 20%, 30%, 40%, or 50% of cells being iPSC precursor cells obtained through mesenchymal-to-epithelial transition (MET), and
(c) harvesting the reprogramming intermediate obtained in step (b), and culturing the reprogramming intermediate in a liquid medium under a 3D suspension culture condition to generate a spheroid of reprogrammed iPSCs, wherein the somatic cell is a non-human primate or human somatic cell.

In some embodiments, the iPSC precursor cells obtained through MET in the reprogramming intermediate lose original morphological characteristic of the somatic cell and are in a MET morphology characterized by domed, tightly packed cell colonies and optionally an increased nuclear-to-cytoplasmic ratio. Preferably, the iPSC precursor cells obtained through MET express a pluripotency marker of TRA-1-60 and/or SSEA3.

In some embodiments, step (c) according to the method of the present invention comprises harvesting the reprogramming intermediate at approximately day 7 to day 21 of reprogramming, more preferably at approximately day 10 to day 18 of reprogramming, for example, harvesting the reprogramming intermediate on day 10, day 11, day 12, day 13, day 14, day 15, day 16, day 17, or day 18.

In some embodiments, the somatic cell for the method of the present invention is a somatic cell or cell line from an adult individual. In some preferred embodiments, the somatic cell is a human blood cell, for example, a human peripheral blood mononuclear cell (PBMC). In some other preferred embodiments, the somatic cell is a human fibroblast.

In some embodiments, in step (c) according to the method of the present invention, the 3D suspension culture is a suspension culture system with stirring or rotating, for example, a roller bottle, an orbital shaker or a bioreactor.

In some embodiments, step (c) according to the method of the present invention further comprises passaging the spheroid of cell obtained after a period of suspension culture for at least one generation, preferably for at least 2 or 3 generations, to obtain a stable cell population of iPSCs.

In some embodiments, the reprogramming factor for the present invention is a reprogramming transcription factor, and step (a) comprises introducing one or more reprogramming factors or a nucleic acid encoding the reprogramming factor into the somatic cell to increase expression or protein amount of the reprogramming factor in the somatic cell.

In some embodiments, step (a) according to the method of the present invention comprises delivering the reprogramming factor to the somatic cell using an integrating virus, a non-integrating virus, an mRNA, an episomal plasmid, or a protein. In some preferred embodiments, step (a) comprises delivering the reprogramming factor to the somatic cell using a non-integrating virus, in particular a Sendai virus vector.

In some embodiments, the one or more reprogramming factors for the present invention comprise or consist of at least three or at least four of Klf4, c-Myc, Oct3/4, Sox2, Nanog and Lin28; preferably, the one or more reprogramming factors comprise or consist of Oct3/4, Sox2, c-Myc and Klf4.

In some embodiments, step (c) according to the method of the present invention comprises culturing the reprogramming intermediate in suspension at a rotating speed of 30 to 100 rpm, preferably at a rotating speed of approximately 70 rpm. Preferably, according to step (c) of the method of the present invention, the 3D suspension culture is carried out in an iPSC maintenance medium (preferably in StemScale PSC suspension medium).

In a second aspect, the present invention provides a method for maintaining or expanding 3D-reprogrammed iPSCs in a 3D culture system. The method can be scaled up for large-scale production of iPSCs.

In some embodiments, the method for maintaining or expanding 3D-reprogrammed iPSCs of the present invention comprises:
(i) obtaining the spheroid of iPSC reprogrammed from the somatic cell according to the 3D reprogramming method of the present invention; and
(ii) maintaining or expanding the iPSC under a 3D suspension culture condition.

In some embodiments, the method further comprises a step of passaging cells after step (i) and before step (ii).

In some embodiments, the passaging cells is performed when the spheroid of iPSC reaches a size of 300 to 500 μm.

In some embodiments, in step (ii) of the method, the maintaining iPSCs comprises passaging cells once every 4 to 6 days, preferably, adding a rho-associated protein kinase (ROCK) inhibitor, preferably Y27632, to a medium within the first 12 to 48 h (preferably 24 h) after passaging cells. In some embodiments, the passaging cells comprises producing single cells or a cell mass consisting of no more than 200 cells from the spheroid of iPSC, and inoculating; preferably, producing a suspension of iPSC single cells from the spheroid of iPSC by enzymatic digestion (for example Accutase enzyme) for passaging cells.

In a third aspect, the present invention provides a method for producing a downstream cell-derived product. The method comprises obtaining 3D-reprogrammed iPSCs by the 3D reprogramming method of the present invention, and producing the downstream cell-derived product by using the iPSCs. Since the 3D reprogramming method of the present invention can provides a high yield of 3D-reprogrammed iPSCs, it is suitable for the large-scale production of the downstream cell-derived product.

### Advantages of the Method of the Present Invention

By the method of the present invention, integrating 3D culture in reprogramming process enables generation of iPSC with high quality.

By the method of the present invention, integrating 3D culture in reprogramming process enables large-scale expansion during the reprogramming process, resulting in a higher yield of iPSCs in a shorter period compared to 2-dimensional reprogramming and culture methods.

### BRIEF DESCRIPTION OF DRAWINGS

FIG. 1 shows the morphology of cells reprogramed by 2D culture as observed under a phase contrast microscope on day 0 (A), day 7 (B), and day 35 (C) of the reprogramming.

FIG. 2 shows the results of morphological evaluation and cell counting of iPSC spheroids obtained by 3D reprogramming; wherein A shows the comparison of representative images of iPSCs generated by 2D and 3D reprogramming methods, and B shows the comparison of iPSCs yield of the 2D and 3D reprogramming methods.

FIG. 3 shows representative images of immunofluorescence staining of iPSCs obtained by 2D reprogramming (top) and 3D reprogramming (bottom) methods; wherein the the pluripotency markers OCT4, SOX2, and TRA-160 were subjected to immunofluorescence staining, and cell nuclei were stained with DAPI. Peripheral blood mononuclear cells (PBMCs) (A) and fibroblasts (B) were used as the starting somatic cell sources for reprogramming.

FIG. 4 shows the relative expression levels of Sendai virus transgenes (A) and pluripotency markers (B) in the 3D-reprogrammed iPSCs, corresponding somatic cell sources, and Sendai virus controls, as measured by RT-PCR.

FIG. 5 shows the trilineage differentiation potential of 3D reprogrammed iPSCs; wherein A shows the differentiation efficiencies of 3D-reprogrammed iPSCs into endoderm, mesoderm, and ectoderm compared to that of 2D-reprogrammed iPSCs, and B shows the representative plots of FACS analysis of endoderm, mesoderm, and ectoderm differentiated from the 2D- and 3D-reprogrammed iPSCs.

### DETAILED DESCRIPTION

### Definitions

Unless otherwise defined, all technical and scientific terms used herein have the same meanings as commonly understood by those of ordinary skills in the art. For purposes of the present invention, the following terms are defined below.

The term “approximately” when used in conjunction with a numerical value is intended to encompass a numerical value ranging from a lower limit that is 5% less than the specified numerical value and an upper limit that is 5% greater than the specified numerical value.

When used in conjunction with two or more options, the expression “and/or” should be understood as meaning any one of the options or any combination of two or more of the options.

As used herein, the term “comprise” or “include” means comprising the involved elements, integers, or steps, but not excluding any other elements, integers, or steps. When the term “comprise” or “include” is used herein, it also encompass situations consisting of the involved elements, integers, or steps, unless otherwise indicated.

As used herein, “expression marker” or “marker” can be used to determine the type of cell. Certain DNA sequences stored with information of cell-specific genes may be transcribed into mRNA and are usually subsequently translated into proteins (gene products) that exert specific function in the cell. The expression of such markers can be detected and quantified at either the RNA or protein level using methods known in the art to characterize the cell. Markers useful in characterizing pluripotent stem cells as well as cell lineages of the three germ layers (mesoderm, endoderm, and ectoderm) are well known in the art.

As used herein, the terms “pluripotent” or “stemness” refers to cells with the ability to produce progeny that can differentiate, under appropriate conditions, into cell types of the three germ layers (endoderm, mesoderm, and ectoderm). Standard assays well-known in the art, for example the ability of teratomas formation in SCID mice aged 8-12 weeks or the trilineage differentiation assay described in examples, can be used to assess the pluripotency of a cell population. Pluripotency of cells can also be assessed by identifying other characteristics of pluripotent stem cells. These characteristics include, but are not limited to, the expression or no expression of combinations of certain molecular markers. Cell markers of human pluripotent stem cells can be one or more (preferably at least four) selected from the following markers: SSEA-3, SSEA-4, TRA-1-60, TRA-1-81, ALP, PODXL, SOX2, E-cadherin, EPCAM, KLF4, LIN-28A, SOX11, SALL2, PRDM14, OCT4 (POU5F1), REX1 and NANOG. Pluripotent stem cells can also be characterized by cell morphology associated with pluripotent stem cells. Typically, pluripotent stem cells on a 2-dimensional matrix grow typically as flat and tightly packed colonies with clear borders. Under a phase contrast microscope, pluripotent cells can be distinguished from somatic cells based on their morphology, for example, fibroblasts display a characteristic elongated spindle shape and have a significantly larger size.

As used herein, the term “an induced pluripotent stem cell” (also referred to as “iPS” cell herein) refers to a cell that is generated from a somatic cell by expressing or inducing the expression of a combination of cytokines, and has the ability to differentiate into various cell types that exhibit characteristics related to cell lineages of the three germ layers (endoderm, mesoderm and ectoderm). Typically, an iPSC (i.e., an iPS cell) has a typical morphology of a pluripotent stem cell, can self-renew, has the trilineage differentiation ability, and expresses a combination of multiple pluripotency markers (such as OCT4, NANOG, SOX2 and PDOXL).

As used herein, the term “somatic cell” refers to any somatic cells of a mammal other than germ line cells (spermatogonia and oocytes). Examples of such cells include differentiated cells, as well as their precursor cells and progenitor cells. Examples of differentiated cells include, but are not limited to, cells from various tissues such as bone marrow, skin, skeletal muscle, adipose tissue, and peripheral blood, exemplarily including but not limited to, fibroblasts, hepatocytes, myoblasts, neurons, osteoblasts, and T cells. In the present invention, any non-pluripotent cell (e.g., a less-differentiated or highly differentiated cell) can be used as the starting somatic cell to generate iPSC. In some embodiments, the somatic cell is a “non-embryonic somatic cell”, meaning that the somatic cell is not present in or not obtained from an embryo, and is not produced by in vitro proliferation of such cells. In some embodiments, the somatic cell is an “adult somatic cell”, meaning that the cell is present in or obtained from a non-embryonic and non-fetal adult individual, or is produced by in vitro proliferation of such cells. For example, the somatic cell may be a human somatic cell from an adult or a human cell from an adult. Somatic cells may be immortalized, for example by increasing the expression of telomerase reverse transcriptase, thereby acquiring the ability to unlimitedly proliferate. In some embodiments, the somatic cell is a differentiated somatic cell or immortalized cell line thereof, including a somatic cell or immortalized cell line thereof from a fetal, neonatal, juvenile, or adult primate individual (including human individual). In some embodiments, the somatic cell used in the present invention is selected from the group consisting of a bone marrow cell, an epithelial cell, an endothelial cell, a fibroblast, a hematopoietic cell, a hepatocyte, a keratinocyte, an intestinal cell, a mesenchymal cell, a myeloid precursor cell, and a splenocyte. In some other embodiments, the somatic cell used in the present invention is a cell that is capable of self-proliferation and differentiation into certain cell types, and the cell is selected from, for example, a heamatopoitic stem cell, a brain stem cell, and a hepatic stem cell. In some embodiments, the somatic cell used in the present invention is selected from the following cells from human or non-human primates: a bone marrow cell, an epithelial cell, an endothelial cell, a fibroblast, a peripheral blood mononuclear cell, a hematopoietic cells, a keratinocyte, a hepatocyte, an intestinal cell, a mesenchymal cell, a bone marrow precursor cell, a splenocyte, and a CD34-positive cell harvested from placental blood. In some preferred embodiments, the somatic cell is a human fibroblast (preferably a skin fibroblast) or a human blood cell (preferably a human PBMC). For different application purposes, such as therapeutic purposes, the somatic cell used in the method of the present invention may be an autologous cell isolated from a human individual to be treated with cell therapy, or an allogeneic cell relative to the individual. Moreover, in some embodiments, the somatic cell may be a cell genetically modified by recombinant techniques. For the purpose of reprogramming according to the present invention, it should be understood that the suitable somatic cell is receptive, or can be made receptive using methods known in the art, meaning that it can uptake reprogramming factors or genetic materials encoding these factors, or can increase the expression of reprogramming factors in cells after subjected to reprogramming treatment. Those skilled in the art are well familiar with the morphological characteristics of various somatic cells and can readily determine the time when reprogramming of somatic cells starts by monitoring the time when the characteristic morphology of somatic cells disappears. The state of the reprogramming of a somatic cell into a pluripotent stem can also be determined by monitoring the upregulation of at least one pluripotency marker.

As used herein, the term “reprogramming” refers to a process of converting a non-pluripotent cell (including a mature, specialized, or differentiated cell) into an induced pluripotent stem cell. Reprogramming is a multi-step and progressive process. Studies have shown that the reprogramming process involves the sequential occurrence of multiple cellular states and can be broadly divided into: initiation phase, maturation phase, and stabilization phase. In the initiation phase, cells under reprogramming express no or very few pluripotency markers and are unstable and reversible. In the maturation phase, these cells express a subset of pluripotency markers. In the stabilization phase, cells under reprogramming express a combination of multiple pluripotency markers and can constantly divide to yield undifferentiated iPSCs. The disappearance of original typical morphology in somatic cells cultured in vitro indicates the onset of reprogramming of somatic cells into a pluripotent state (See Philos Trans R Soc Lond B Biol Sci. 2018 Jul. 5; 373 (1750): 20170213, “Pluripotent stem cells: induction and self-renewal”, doi: 10.1098/rstb.2017.0213).

As used herein, the term “reprogramming factor” refers to a factor or a combination of factors capable of promoting the reprogramming of a somatic cell into a pluripotent state. Typical reprogramming factors are transcription factors having a reprogramming function and are also referred to herein as “reprogramming transcription factors” or “transcription factors”. Transcription factors useful in reprogramming are known in the art (See, for example https://doi.org/10.1038/nrm.2016.8 (“A decade of transcription factor-mediated reprogramming to pluripotency”)). Some examples of common reprogramming transcription factors and their Ensemble Gene IDs and Uniprot IDs are shown in the table below, from which relevant nucleotide sequence and amino acid sequence information can be obtained. Functional variants, homologs, orthologs and paralogs of these factors also should be taken into consideration by the present invention.

TABLE A
Some exemplary reprogramming transcription factors

| | | Uniprot |
|---|---|---|
| Transcription factor | Ensemble Gene ID | ID |
| OCT4 (also known as POU5F1) | ENSG00000204531 | Q01860 |
| SOX2 | ENSG00000181449 | P48431 |
| cMYC | ENSG00000136997 | P01106 |
| KLF4 | ENSG00000136826 | O43474 |
| LIN28 | ENSG00000131914 | Q9H9Z2 |
| NANOG | ENSG00000111704 | Q9H9S0 |

As used herein, the term “reprogramming treatment” means subjecting a somatic cell to a condition that allows reprogramming of the somatic cell into an induced pluripotent stem cell. Various methods for reprogramming somatic cell are known in the art. Typically, the reprogramming of a somatic cell involves increasing the amount of a reprogramming factor (for example, a reprogramming transcription factor) in a somatic cell and subsequently culturing the somatic cell under a specific condition to promote loss of differentiation markers and acquisition of pluripotency markers. As used herein, the term “day 0 of reprogramming” refers to the day on which a somatic cell begins to receive reprogramming treatment, for example transgenesis of reprogramming factors via transfection or transduction.

As used herein, in cases where the reprogramming factor is a transcription factor, the phrases “increasing the amount of the reprogramming factor” and “increasing the expression or protein amount of the reprogramming factor” can be used interchangeably. These phrases indicate an increase in the expression level and/or the amount of expressed product of the reprogramming factor (e.g., a reprogramming transcription factor) in a target cell (for example a somatic cell subjected to reprogramming treatment, such as a fibroblast or PBMC), relative to a control cell (i.e., the corresponding cell without reprogramming treatment). In some embodiments, the amount of a reprogramming transcription factor in a target cell (e.g., a somatic cell into which an exogenous nucleic acid encoding the transcription factor was introduced) is increased by at least 10%, 20%, 30%, 40%, 50%, 60%, 70%, 80%, 90%, or more, relative to a control cell (e.g., the corresponding somatic cell into which no exogenous nucleic acid was introduced). Any method for increasing the amount of the transcription factor should be taken into consideration by the present invention, including methods for increasing the transcription level, transcription rate, or transcription efficiency of the transcription factor, enhancing translation of the transcription factor and increasing corresponding protein production, and increasing the stability or activity of the transcription factor in a cell.

As used herein, the term “exogenous” related to a protein, gene, nucleic acid or polynucleotide in a cell or organism means that the protein, gene, nucleic acid or polynucleotide is artificially introduced into the cell or organism. Moreover, when an endogenous counterpart of the protein, gene, nucleic acid or polynucleotide is present in the cell or organism, the introduced protein, gene, nucleic acid or polynucleotide is present in a form differing from the endogenous counterpart (for example located in a different position, or in an unnatural nucleic acid construct, or linked to a different flanking nucleic acid sequence) in the cell or organism. Thus, a transcription factor (e.g. SOX2) introduced into a target somatic cell through recombination is considered to be “exogenous” with respect to that cell, differing from the corresponding naturally occurring transcription factor found in such somatic cell (e.g. endogenous SOX2). In some embodiments, the exogenous nucleic acid is present in a target cell in an extrachromosomal form, e.g., a form of episomal vector.

As used herein, Kruppel-like factor 4 (abbreviated as KLF4), also known as GKLF, is a member of the Kruppel-like factor (KLF) family of zinc finger transcription factors. KLF4 is involved in the regulation of cell proliferation, cell differentiation, apoptosis, and reprogramming of somatic cell. It plays an important role in maintaining the stemness of embryonic stem cells and preventing their differentiation. An example of KLF4 can be found under UniProt accession number: 043474.

As used herein, Oct4, also called as POU5F1 or Oct3/4, is a transcription factor encoded by the POU5F1 gene. Oct4 contains a POU-homology domain and is critical for self-renewal and maintenance of stemness in undifferentiated embryonic stem cells. Oct4 is also commonly used as a molecular marker for identifying undifferentiated cells. An example of Oct4 can be found under UniProt accession number: Q01860.

As used herein, SOX2 is a member of the SRY-related HMG (high mobility group) box (SOX) transcription factors. The transcription factor SOX2 can form a trimeric complex with OCT4, which can control the expression of numerous genes involved in embryonic development and thereby plays a role in regulating embryonic development and in cell fate determination. An example of SOX2 can be found under UniProt accession number: P48431.

As used herein, PODXL (also known as podocalyxin) is a negative charge-rich sialomucin protein that belongs to the CD34 protein family. PODXL carries many post-translational epitopes responsible for various surface pluripotency markers, including TRA-1-60, TRA-1-81, GCTM2, GP200, and mAb84.

As used herein, “Nanog” refers to the homeobox protein NANOG, which is a transcriptional regulator involved in the proliferation and self-renewal of the inner cell mass and embryonic stem (ES) cells. An example of NANOG can be found under UniProt accession number: Q9H9S0.

As used herein, “Lin28” refers to an RNA-binding protein found in embryogenesis, including two paralogs: LIN28A and LIN28B. It has been reported that somatic cells can be reprogrammed to have pluripotency using a combination of OCT4, SOX2, NANOG, and either LIN28A or LIN28B. An example of LIN28A can be found under UniProt accession number: Q9H9Z2, and an example of LIN28B can be found under UniProt accession number: Q6ZN17. As used herein, “KOS” refers to a polycistronic transgene encoding reprogramming factors of hKLF4, hOct3/4 and hSox2, which is presented in a single vector (e.g., a Sendai virus vector). In reprogramming systems utilizing a Sendai virus vector containing KOS, an additional hKLF4-expressing viral vector may be added to compensate for insufficient viral polymerase activity caused by the polycistronic configuration of KOS, thereby enhancing reprogramming efficiency.

As used herein, “c-Myc”, as a member of the Myc family, is a proto-oncogene and regulator gene that encodes a transcription factor. POU5F1, SOX2, c-Myc, and KLF4 are known as the four Yamanaka transcription factors. It has been demonstrated that these four factors, when combined, are sufficient to reprogram differentiated cells into induced pluripotent stem (iPS) cells. An example of c-Myc can be found under UniProt accession number: P01106.

As used herein, the term “Tra-1-60” refers to a monoclonal antibody Tra-1-60, or depending on context, refers to the Tra-1-60 antibody-specifically recognized carbohydrate epitope pluripotency marker on PODXL. In some embodiments of the present invention, the expression of this epitope, as detected by the Tra-1-60 antibody, can be used to assess the pluripotent state of a 3D reprogramming intermediate.

As used herein, “SSEA3” refers to stage-specific embryonic antigen 3, which is a cell-surface glycosphingolipid antigen associated with pluripotent stem cells.

As used herein, the term “mesenchymal-to-epithelial transition” (abbreviated as MET or MET transition) refers to an important event that occurs early in the reprogramming of somatic cells into induced pluripotent stem cells (iPSCs) in vitro. After this event, cells undergoing reprogramming obtain stemness or pluripotency through mesenchymal-to-epithelial transition. As used herein, compared to the MET, the term “epithelial-to-mesenchymal transition” (abbreviated as EMT) refers to an event of epithelial-to-mesenchymal transition of somatic cells that occurs prior to the MET in the reprogramming of somatic cells into induced pluripotent stem cells (iPSCs) in vitro.

As used herein, the terms “MET morphology” or “MET phenotype” refer to that after undergoing MET, cells lose the original morphology of the somatic cells during the in vitro reprogramming, and the cells in 2D-culture show a morphology or phenotype different from typical iPSC morphology and characterized by domed, tightly packed cell colonies and optionally an increased nuclear-to-cytoplasmic ratio.

As used herein, the term “iPSC morphology” or “iPSC phenotype” means that cells under reprogramming show typical morphology of iPSCs cultured in vitro, and the cells in two-dimensional culture show tightly packed, flat multi-cell colonies with well-defined borders. The cells in colonies are round and exhibit a high nucleus-to-cytoplasm ratio, with prominent nucleoli. Healthy iPSC colonies will merge together seamlessly, and there will be multilayers of cells in the center, resulting in dense clusters of cells as observed under a phase contrast microscope. However, as known in the art, iPSC colonies may display slightly different morphologies depending on the different substrates used for growth. For example, colonies grown on Vitronectin XF™ may appear more compact and round, whereas those grown on Corning® Matrigel® may be relatively loosely distributed and irregularly shaped. Spontaneous differentiation of iPSCs on the two substrates is characterized by loss of colony border integrity, the appearance of regions with irregular cell morphology within a single colony, or the appearance of other cell types.

As used herein, the term “reprogramming intermediate” refers to a cell culture under reprogramming that has not yet fully induced to form stable iPSC colonies.

As used herein, the term “reprogramming intermediate obtained through MET” (or simply “MET reprogramming intermediate”), means that such a reprogramming intermediate comprises at least 20%, 30%, 40%, or 50% iPSC precursor cells obtained through MET, thereby exhibiting a MET morphology or MET phenotype. In 2-dimensional culture, morphological changes of cells under reprogramming can be monitored using phase contrast microscope to identify un-reprogrammed original somatic cells, reprogramming intermediates obtained through MET and stable iPSCs. Cells under reprogramming will acquire stemness or pluripotency after MET and express a subset of pluripotency markers. Thus, changes in expression levels of one or more specific pluripotency markers as well as transgenic reprogramming factors in cells under reprogramming can also be monitored and optionally compared with that of un-reprogrammed original somatic cells and stable iPSCs to identify MET reprogramming intermediates. In some preferred embodiments, the MET reprogramming intermediate comprises at least 20%, 30%, 40%, or 50% of reprogrammed cells having an increased expression of pluripotency markers TRA-1-60 and/or SSEA3 as compared to the original somatic cells. Preferably, there are less than 20%, 30%, 40% or 50% of reprogrammed cells being positive to comprise at least four pluripotency markers of SOX4, PODXL, OCT4, and NANOG, and/or having an expression level of transgenic reprogramming transcription factors higher than that of stable iPSCs. Generally, for human somatic cells, somatic cells under reprogramming in vitro enter MET at day 5 to day 21 of reprogramming. However, as can be understood by one of skill in the art, the specific time of entry into MET will depend on the type of somatic cells used and the specific culture conditions. For human fibroblasts, the MET reprogramming intermediate suitable for 3D suspension culture of the present invention can be obtained, for example, at day 10 to day 21, preferably at approximately day 13 to day 18.

As used herein, the term “MET rate” means the percentage of cells that exhibit a MET morphology or phenotype after the reprogramming intermediate undergo MET. Preferably, the cells obtained through MET also express one or both of the pluripotency markers of TRA-1-60 and SSEA3. The MET rate of reprogramming intermediate for 3D suspension culture affects iPS cell yield. Although not wishing to be bound by theory, it is believed that when the MET rate is too low, the reprogramming intermediate from human cells is very unstable, resulting in reduced efficiency to obtain totipotent iPS cells. The MET rate of reprogramming intermediate suitable for 3D suspension culture can be determined by one of skill in the art based on the type of somatic cell used and the culture conditions according to the teachings of the present invention. In some embodiments, the MET rate suitable for the method of the present invention is at least 20%, 30%, 40%, or 50%. In some other embodiments, the MET rate suitable for the method of the present invention is at least 50%, 55%, 60%, 65%, 70%, 75%, or 80%.

As used herein, the term “adhered to wall” or “attached to wall” means that cells are attached to the surface of a vessel. For example, in the presence of appropriate medium, cells are attached to the surface of a sterile plastic culture dish or flask, or a sterile culture dish or flask coated with plastic.

As used herein, the two-dimensional (2D) culture refers to a cell culture method that allows cells to attach to the wall and grow in two dimensions to form a monolayer of cells. In the 3D reprogramming method of the present invention, somatic cells should be cultured under a 2D culture condition for at least a period to enter the MET state before starting 3D culture. 2D culture methods and conditions suitable for reprogramming various human somatic cells are well known in the art and can be applied to the method of the present invention to obtain the MET reprogramming intermediate of the present invention. For example, somatic cells to be induced into a pluripotent state may be cultured on feeder layer cells (e.g., MEF), or may grow by directly attaching to a solid culture surface (e.g., a culture plate). Media suitable for culturing various cell types are well known in the art and commercially available. For instance, fibroblasts can be cultured using Essential 6 or Essential 8 medium from Thermo Fisher Scientific.

As used herein, the term “three-dimensional culture (also referred to as 3D culture)” refers to a cell culture method that allows cells grow in three dimensions to form cell mass/clusters or specific tissue structures. Typical types of 3D culture include those that rely on, and those that do not rely on, solid or semi-solid supports. 3D culture methods that rely on supports include, for example, those that use a pre-prepared three-dimensional scaffold (usually polymer scaffold) on which cells are inoculated and allowed to grow. 3D culture methods that do not rely on supports include hanging drop culture, culture with low-attachment microplates, and culture with rotating bioreactors.

As used herein, the term “3D suspension culture” means culturing single cells or small cell mass with constant stirring in a liquid medium to allow cells to proliferate in a non-adherent manner and grow in three dimensions.

As used herein, the terms “stable iPSC” and “fully reprogrammed cell” can be used interchangeably in the present invention to refer to an iPSC that exhibits the full characteristics of embryonic stem (ES) cells and is capable of unlimited division into undifferentiated iPSCs. Since reprogramming is a complex process that includes multiple events, only a small fraction of cells can successfully complete it. Partially reprogrammed cells that do not fully complete the process may also exhibit ES-like morphology, but their epigenetic state retains traces of somatic cell characteristics, and may subsequently undergo differentiation or death (See, for example, https://doi.org/10.1038/nrm.2016.8). Therefore, it usually still takes several cell cycles for iPSC precursor cells obtained through MET to establish stable iPSC colonies. The expression or expression level of pluripotency markers in cells under reprogramming can be used to distinguish stable iPSCs, cells in the early stage of reprogramming (almost no expression of pluripotency markers) and cells in the mature stage of reprogramming (only expression of a subset of pluripotency markers). Furthermore, it has also been recognized that although the expression of exogenous reprogramming factors should persist for a sufficient period during reprogramming, the generation and stabilization of iPSCs depends on the silence of the transgenic reprogramming factors at a time point of the later stage of reprogramming approximately when the cell fate is directed to an ES-like state. Thus, the stability of iPSC colonies can also be examined by monitoring the silence of such transgenes. Typically, the stable iPSCs can self-renew, produce various cell types of three germ layers, express pluripotency markers OCT4, NANOG, SOX2 and PODXL, and have the silence of transgenic reprogramming factors. As used herein, the term “stable iPSC population” or “stable iPSC colony” means that at least 90% or more of cells in the cell population or colony are stable iPSCs that present typical morphological characteristics of iPSC and express markers of pluripotent stem cells.

As used herein, the term “two-dimensional conventional reprogramming procedure” refers to a two-dimensional cell culture procedure that relies on adherent culture and cell passaging and begins with the reprogramming of somatic cell and ends with the generation of stable iPSCs. Taking fibroblasts and PBMCs as examples, typically, such procedure involving transfecting nucleic acids encoding reprogramming factors comprises the following sequential steps: introducing the reprogramming factors into somatic cells under adherent culture via transduction or transfection on the day of transfection (day 0 of reprogramming), culturing for several days (e.g., approximately 5-7 days), and inoculating cells into a new culture plate for 1-4 weeks or longer of continuous adherent culture until cell colonies appear and grow to a size suitable for passaging. During this reprogramming process, typically, initial cell colonies will appear in the inoculated culture plate a few days after transfection (e.g., 5-12 days). As cells grow, the size of the colony gradually increases until it covers a major area of the culture vessel's surface. However, only a small fraction of these colonies contain fully reprogrammed cells. It is difficult to distinguish colonies containing partially reprogrammed cells from those containing fully reprogrammed cells by observation with naked eyes. Therefore, in conventional 2D-reprogramming procedures for iPSC generation, after the initial colonies are obtained, subsequent selection of iPSC colonies and/or multiple rounds of cell passaging in two-dimensional culture are usually required to ensure the growth of real iPSCs and remove un-reprogrammed cells, dead cells, differentiated cells, and other non-pluripotent cells, thereby yielding a “stable” iPSC culture.

As used herein, 2D-iPSC refers to an iPSC obtained through a conventional 2D reprogramming procedure. 3D-iPSC, as used herein, refers to an iPSC obtained through the 3D reprogramming procedure of the present invention.

As used herein, the term “downstream cell-derived product” refers to iPSCs generated from reprogramming, or other iPSCs or differentiated cells generated from proliferation and differentiation, such as cardiomyocytes, macrophages, hepatocytes, neuronal cells, and precursor cells thereof. Such a cell-derived product can be transferred to a mammalian individual that can serve as the somatic cell source for generation of these iPSCs. In other words, the somatic cell for use in the method of the present invention may be an autologous cell. Alternatively, the somatic cell for use in the method of the present invention may be an allogeneic cell with respect to the mammalian individual to which the downstream cell-derived product is administered. Preferably, the somatic cell for use in the method of the present invention is derived from a patient to be treated with the cell-derived product, i.e., an autologous cell.

Various aspects of the present invention are described in further detail below.

### 3-Dimensional Reprogramming Method of the Present Invention

In the conventional 2D-reprogramming processes for iPSC generation, it is generally believed that passaging the initial colonies appeared after reprogramming too early makes the cell mass non-adherent. This affects the iPSC yield and may result in cell differentiation, leading to the appearance of other cell types and different morphologies. However, the inventors of the present invention, through devoting themselves to studies on reprogramming human somatic cells and 3D culture, surprisingly find that introducing 3D suspension culture into the relatively early stage of 2D-reprogramming when mesenchymal-to-epithelial transition (MET) of human somatic cells occurs can effectively integrate reprogramming with 3D culture to obtain stable iPSC generation and proliferation. Based on this finding, the inventors of the present invention develop the 3D reprogramming method of the present invention useful in the reprogramming of human somatic cells as well as non-human primate somatic cells.

Accordingly, in a first aspect, the present invention provides a method for producing an iPSC by reprogramming a human or non-human primate somatic cell, comprising steps of:
(a) performing reprogramming treatment on a human somatic cell to increase expression or protein amount of one or more reprogramming factors in the somatic cell,
(b) culturing the somatic cell treated in step (a) under a 2D culture condition for a period sufficient to obtain a reprogramming intermediate, wherein the reprogramming intermediate is characterized by at least 20%, 30%, 40%, or 50% of cells being iPSC precursor cells obtained through mesenchymal-to-epithelial transition (MET), and
(c) harvesting the reprogramming intermediate obtained in step (b), and culturing the reprogramming intermediate in a liquid medium under a 3D suspension culture condition to generate a spheroid of reprogrammed iPSCs. In the following description, taking a human somatic cell for example, aspects of the present invention are illustrated. It should be understood that these aspects of the present invention also encompass any embodiment in which a human somatic cell is replaced with a non-human primate somatic cell.

### Reprogramming Intermediate

The terms mesenchymal-epithelial transition (MET) and epithelial-mesenchymal transition (EMT) are well-known in the art. It is known in the art that differentiated somatic cells can be reprogrammed into pluripotent cells in vitro through sequential EMT-MET events. Blocking MET in the early stage of reprogramming of somatic cell will reduce the efficiency of reprogramming (https://doi.org/10.1038/s41556-018-0195-z, “Mesenchymal-epithelial transition in development and reprogramming”, 2019). The reprogrammed cells obtained through MET show characteristic changes in gene expression related to MET, such as the downregulation of mesenchymal-related genes and/or upregulation of epithelial-related genes. Additionally, the cytoskeleton is remodeled during MET, leading to the loss of the original morphology of somatic cells. In 2D in vitro culture, after undergoing MET, somatic cells present characteristic MET morphology or phenotype, which exhibits as domed and tightly packed colonies of cells different from that of typical iPSCs, where the cells are round and have a high nuclear-to-cytoplasmic ratio similar to iPS cells, but have larger cell sizes.

According to the method of the present invention, the reprogramming intermediate showing such MET morphology or MET phenotype can facilitate the proliferation in three dimensional suspension culture and generation of stable spheroids of iPSCs. Thus, in some embodiments, the method of the present invention comprises inoculating the reprogramming intermediate (i.e., the MET reprogramming intermediate) cultured in two dimensional culture into a liquid medium and culturing the reprogramming intermediate under a 3D suspension culture condition for a period sufficient to obtain a spheroid of iPSCs.

In some embodiments, the reprogramming intermediate according to the present invention may be a mixture of partially reprogrammed cells, fully reprogrammed cells, unprogrammed cells and/or differentiated cells. In some embodiments, the reprogramming intermediate for use in the method of the present invention has a MET rate of at least 20%, 30%, 40% or 50%, wherein the percentage of reprogramming cells are iPSC precursor cells obtained through MET. In some embodiments, the reprogramming intermediate has a MET rate of at least 50%, 55%, 60%, 65%, 70%, 75%, 80%, 85% or 90%. In some preferred embodiments, the reprogramming intermediate has a MET rate of 20-80%. In some other preferred embodiments, the reprogramming intermediate has a MET rate of 50-90%. In yet other preferred embodiments, the reprogramming intermediate has a MET rate of 60-80%.

In some embodiments, the iPSC precursor cells obtained through MET in the reprogramming intermediate present a MET morphology or a MET phenotype. Preferably, they also express one or both of the pluripotency markers TRA-1-60 and SSEA3.

The time point for harvesting a suitable MET reprogramming intermediate may vary depending on the type of somatic cells. In some embodiments, the MET reprogramming intermediate is harvested at approximately day 7 to day 21 of reprogramming, for example, harvesting the MET reprogramming intermediate on day 10, day 11, day 12, day 13, day 14, day 15, day 16, day 17 or day 18. In some embodiments, the MET reprogramming intermediate is harvested at approximately day 10 to day 21, approximately day 13 to day 18 or approximately day 13 to day 15 of reprogramming. In some embodiments, the somatic cell is a human fibroblast or a human blood cell, and the MET reprogramming intermediate is harvested at approximately day 13 to day 21 of reprogramming, preferably at approximately day 13 to day 18 or approximately day 13 to day 15 of reprogramming.

### Delivery of Reprogramming Factor

In order to obtain the MET reprogramming intermediate of the present invention, reprogramming treatments known to the present invention may be applied to increase the expression or protein amount of the reprogramming transcription factor in the target somatic cell for transformation purposes.

The pluripotency induction was initially achieved by reprogramming of mouse somatic cells in 2006 (Yamanaka et al., 2006) and human somatic cells in 2007 (Yu et al., 2007; Takahashi et al., 2007) through introducing transcription factors associated with pluripotency. Recently, the use of various small molecular reprogramming factors has also been proposed (See https://doi.org/10.1038/s12276-020-0383-3, 2019, “Small-molecule-mediated reprogramming: a silver lining for regenerative medicine”). Methods for reprogramming human somatic cells to induced pluripotent stem cells (iPSCs) include, but are not limited to, methods described in the following documents: U.S. patent application No. 20090246875, U.S. Patent Publication No. 2010/0210014, U.S. Patent Publication No. 20120276636, U.S. Pat. Nos. 8,058,065, 8,129,187, 8,268,620, PCT Publication WO 2007/069666 A1 and U.S. Pat. No. 8,268,620, and they are hereby incorporated herein by reference. All of the above methods of pluripotency induction are useful in the method of the present invention to obtain the reprogramming intermediate of the present invention.

In some embodiments, accordingly, step (a) of the method of the present invention comprises subjecting a human somatic cell to reprogramming treatment by contacting the human somatic cell with a reprogramming factor capable of inducing pluripotency. In some embodiments, the reprogramming factor is a small molecular compound. In some other embodiments, the reprogramming factor is a transcription factor. In yet another embodiments, the small molecular reprogramming factor is used in combination with a reprogramming transcription factor in the method of the present invention to increase the efficiency of reprogramming. Exemplary small molecular reprogramming factors include, but are not limited to: agents that inhibit H3K9 methylation or promote H3K9 demethylation, agents that inhibit H3K4 demethylation or promote H3K4 methylation, agents that inhibit histone deacetylation or promote histone acetylation, L-type calcium channel agonists, activators of cAMP pathway, DNA methyltransferase (DNMT) inhibitors, nuclear receptor ligands, GSK3 inhibitors, MEK inhibitors, TGFβ receptor/ALK5 inhibitors, HDAC inhibitors, Erk inhibitors, ROCK inhibitors, FGFR inhibitors, and PARP inhibitors. In some other embodiments, the small molecular reprogramming factor is selected from the group consisting of a GSK3 inhibitor, a MEK inhibitor, a TGFβ receptor/ALK5 inhibitor; a HDAC inhibitor, an Erk inhibitor and a ROCK inhibitor.

In some typical embodiments, the reprograming method according to the present invention uses a reprogramming transcription factor for reprogramming a somatic cell to a pluripotent stem cell. In such embodiments, a somatic cell is reprogrammed into a dedifferentiated or pluripotent state, which typically involves increasing the expression or protein amount of the reprogramming factor (including transcription factor) in the somatic cell. Any transcription factor associated with pluripotency may be used as a reprogramming factor according to the present invention. The transcription factor associated with pluripotency may comprise, consist of or consist essentially of one or more of: Oct-3/4, Cdx-2, Gbx2, Gsh1, HesX1, HoxA10, HoxA11, HoxB1, Irx2, Isl1, Meis1, Meox2, Nanog, Nkx2.2, Onecut, Otx1, Oxt2, Pax5, Pax6, Pdx1, Tcf1, Tcf2, Zfhx1b, Klf-4, Atbf1, Esrrb, Genf, Jarid2, Jmjdla, Jmjd2c, Klf-3, Klf-5, Mel-18, Myst3, Nacl, REST, Rex-1, Rybp, Sall4, Sall1, Tifl, YY1, Zeb2, Zfp281, Zfp57, Zic3, Coup-Tf1, Coup-Tf2, Bmil, Rnf2, Mtal, Pias1, Pias2, Pias3, Piasy, Sox2, Lefl, Sox15, Sox6, Tcf-7, Tcf711, c-Myc, L-Myc, N-Myc, Hand1, Mad1, Mad3, Mad4, Mxil, Myf5, Neurog2, ngn3, Olig2, Tcf3, Tcf4, Foxc1, Foxd3, BAF155, C/EBPB, mafa, Eomes, Tbx-3, Rfx4, Stat3, Stella and UTF-1. The mammalian and human cDNA sequences and amino acid sequences of these reprogramming factors can be found in the databases Ensemble GENE and UniProtKB. In some embodiments, the transcription factor used for the method of the present invention comprises, consists of, or consists essentially of one or more of: OCT4, SOX2, KLF4 and MYC (OSKM); SOX2, KLF4 and OCT4 (SKO); OCT4, SOX2, KLF4 and GLIS1 (OSKG); OCT4, SOX2, NANOG and LIN28 (OSNL); or OCT4, SOX2, KLF4, c-MYC, NANOG and LIN28 (OKSMNL). In some preferred embodiments, the transcription factor comprises, consists of, or consists essentially of one or more of: OCT4, SOX2, KLF4 and MYC, preferably, all four factors (OSKM) or variants thereof.

In some embodiments, step (a) of the method of the present invention comprises subjecting a human somatic cell to a reprogramming treatment to increase the expression or protein amount of one or more reprogramming transcription factors in the somatic cell. In some embodiments, the reprogramming treatment comprises introducing a combination of multiple reprogramming transcription factors, or one or more polynucleotides encoding the combination into a target somatic cell. In embodiments where the polynucleotide is used, it can be understood that one or more expression cassettes can be used to achieve the expression of the combination of transcription factors. For example, in case that a single expression cassette is used to express multiple polypeptides, a polycistronic expression cassette can be used. It can also be understood that one or more vectors carrying the polynucleotide or expression cassette can be used to introduce the one or more polynucleotides, or one or more expression cassettes into a target somatic cell. Exemplary vectors include, but are not limited to, viral and non-viral vectors. When viral vectors are used for reprogramming, the viral vectors are preferably non-integrating viral vectors or replication-defective viral vectors (e.g., viral vectors in which nucleic acids encoding essential elements for viral replication or all of the viral nucleic acids are removed). In addition, non-viral vectors such as plasmids, liposomes, nanoparticles, and other means of DNA molecule delivery are also be encompassed within the scope of the present invention. In some embodiments, the present invention uses a viral vector to deliver the reprogramming factor to a target somatic cell. For this purpose, a nucleic acid construct containing a nucleic acid encoding the reprogramming factor can be inserted into the genome of a non-replicative or replication-defective virus and packaged into infectious viral particles. After the viral vector is transfected or transduced into cells, the viral genome containing the construct will enter the cells and, depending on the type of virus, may or may not be integrated into the genome of these cells.

As used herein, the term “non-integrating viral vector” refers to a viral vector that is incapable of integrating its carried genetic material into the genome of the host cell. After a host cell is infected with the viral vector, the genome of such vial vector presents as a non-replicative episome in the transduced cell, thereby avoiding the risk of insertional mutagenesis caused by genomic integration. Exemplary non-integrating viral vectors include, but are not limited to, adenoviruses, adeno-associated viral (AAV) vectors, Sendai virus, poxviruses, and modified non-integrating lentiviral vectors. Viral vectors can be constructed or modified according to methods well known in the scientific literature to enhance their efficiency and safety in reprogramming applications. For example, specific mutations, such as temperature-sensitive mutations, can be introduced into some genes of the viral genome, so as to control the persistence period of the viral vector or silence of expression of the transgenes carried by the viral vector in the host by regulating the culture temperature.

In some embodiments of the method of the present invention, nucleic acids encoding the transcription factors required for reprogramming are delivered into somatic cells via one or more vectors. Each transcription factor may be introduced into the somatic cells in a form of a nucleic acid construct comprising transgenic polynucleotides encoding the factors efficiently linked to a heterologous promoter. A strong constitutive promoter in the form of polycistron can be used to express multiple transcription factors. Expression cassettes under the control of separate promoters can also be used to express the multiple transcription factors on a single vector or separate multiple vectors. Heterologous promoters useful in the method of the present invention can be selected based on the desired expression level of transcription factors and/or the desired silence rate of the promoter. In some embodiments of the present invention, for the method, at least 3 or 4 transcription factors are introduced to the somatic cells. Depending on the type of factors delivered and the type of somatic cells, those skilled in the art can test and readily determine the optimal delivery ratio of factors.

In some embodiments, the nucleic acid sequence encoding the transcription factor protein is introduced into a somatic cell via a plasmid. In some embodiments, the plasmid containing nucleic acids encoding one or more transcription factors may be an episomal plasmid. In an embodiment, the nucleic acid sequences encoding the transcription factor proteins are introduced into the somatic cells via a vector (e.g., a viral or non-viral vector). Preferably, the vector is a viral vector, such as a retroviral vector, lentiviral vector, adenoviral vector, or Sendai virus vector. Preferably, the vector is a non-integrating viral vector. In some embodiments, the expression or protein amount of the reprogramming transcription factors in the somatic cells is increased by transducing or transfecting the somatic cells with the vector.

According to the method of the present invention, an exogenous nucleic acid is preferably used to increase the expression or protein amount of the reprogramming factors in the somatic cells. In addition, a transcriptional activation system (e.g. the CRISPR/Cas9 system for gene expression regulation) can also be used to increase the expression of one or more transcription factors in the somatic cells.

In some preferred embodiments according to the method of the present invention, in step (a), the reprogramming factor is delivered using a Sendai virus (also abbreviated as SeV), which is an attractive vector that is capable of delivering reprogramming factors (including, but not limited to, OCT4, SOX2, KLF4 and CMYC (OKSM)) into somatic cells to generate iPSCs. This virus is non-pathogenic to humans and has an RNA genome that can persist in the cytoplasm, thus eliminating the risk caused by the integration of reprogramming factor or viral gene into the genome. In addition, the use of Sendai virus vectors in the reprogramming of somatic cells to iPSC is well established. Commercial kits for this purpose are readily available, including, but not limited to, CytoTune™-iPS Sendai Virus Reprogramming Kit from Invitrogen. The long-term presence of reprogramming factors and reprogramming vectors in iPSCs is not desirable due to their potential oncogenic risks and their impacts on iPSC analysis. However, advantageously, the Sendai virus vector, as a non-integrating vector, will generally disappear after several passages as cell passaging, thereby preventing the long-term presence of reprogramming factors in iPSCs. Moreover, temperature-sensitive Sendai viruses have now been developed. After the reprogramming factors are introduced into the somatic cells to be reprogrammed, the residual reprogramming vector and transgene expression can be eliminated or silenced in the self-induced iPSCs by changing the temperature at the required stage when needed. To monitor the acquisition of vector-free iPSCs, in some embodiments, a combination of primers of Sendai virus genome sequence and transgene-specific primers can be designed to specifically detect the expression level of transgenes carried by the Sendai virus vector.

The somatic cells suitable for reprogramming are not particularly limited. Generally, differentiated somatic cells, including cells from fetal, neonatal, juvenile or adult mammals, such as primates (including human), are applicable as somatic cells for the method of the present invention. Such somatic cells can be reprogrammed into induced pluripotent stem cells through the three-dimensional reprogramming method of the present invention. In some embodiments, the somatic cell according to the present invention is selected from the group consisting of a bone marrow cell, an epithelial cell, an endothelial cell, a fibroblast, a peripheral blood mononuclear cell, a hematopoietic cell, a keratinocyte, a hepatocyte, an intestinal cell, a mesenchymal cell, a bone marrow precursor cell and a splenocyte. In some more preferred embodiments, the somatic cell is a human fibroblast or peripheral blood mononuclear cell (PBMC).

In some particularly preferred embodiments according to the method of the present invention, the reprogramming treatment according to the present invention comprises transducing a human somatic cell (e.g., human PBMC and fibroblast) with Sendai vectors encoding KOS, hc-Myc, and hKlf4 at a multiplicity of infection ratio of preferably 1:1:0.1-1.5 (such as 5:5:3 or 5:5:6).

### Two-Dimensional Culture for Obtaining Reprogramming Intermediate

After reprogramming treatment (e.g., after obtaining cells transduced with reprogramming factors), the method according to the present invention further comprises culturing the cell obtained through the reprogramming treatment under a two-dimensional culture condition for an appropriate period to obtain a suitable reprogramming intermediate (i.e., step (b) of the method of the present invention). The period of the two-dimensional culture may vary depending on factors such as the source of the somatic cells used and the culture conditions. For example, for human fibroblasts and PBMCs, it can be approximately 7-21 days.

There are no particular limitations to the two-dimensional culture methods applicable to step (b) of the method of the present invention. For example, cells that received the reprogramming treatment in step (a) of the present invention can be cultured according to any two-dimensional culture method for reprogramming known in the art.

In some embodiments, the cells under reprogramming are cultured in contact with feeder cells. Exemplary feeder cells include, but are not limited to: fibroblasts, e.g., mouse embryonic fibroblasts (MEF). Methods of culturing cells on feeder cells are known in the art.

In some embodiments, the cells under reprogramming are cultured under a condition of lacking feeder cells. The cells can, for example, be attached directly to a solid culture surface (e.g., a culture plate), via an artificial matrix. Exemplary matrix includes, but is not limited to: laminin, fibronectin, vitronectin, proteoglycan, nestin, collagen, collagen I, collagen IV, collagen VIII, heparan sulfate, Matrigel™ or any combination thereof. However, it is clear to one of skill in the art that this is a non-limiting list, and other molecules may be used to attach the cells to the solid surface. Methods of initially attaching a matrix to a solid surface are known in the art.

In some embodiments involving the transfection or transduction of nucleic acids encoding reprogramming factors into somatic cells, steps (a) and (b) of the method of the present invention comprise the following operations in sequence:
inoculating the somatic cells into a culture vessel (e.g., a 6-well plate) approximately several days (depending on the type of the somatic cells, e.g., approximately 2-3 days) before transfection or transduction, and culturing the cells to ensure they fully attach to the wall and spread;
on day 0 of reprogramming (i.e., the day of transfection), introducing the nucleic acids encoding reprogramming factors into the somatic cells through transduction or transfection, and culturing the transfected cells in the medium of somatic cells for several days (depending on the type of somatic cells, e.g., approximately 5-10 days); and
inoculating the cells to a culture plate with or without feeder cells for adherent culture, during which (e.g., 2 days after the inoculation), replacing the medium with iPSC medium, and culturing the cells continuously until colonies with MET morphology appearing;
optionally, detecting the cell colonies to obtain MET reprogramming intermediate.

### Three-Dimensional Culture of Reprogramming Intermediate

When the majority (e.g., at least 50% or 70%) of the cell populations in the reprogramming intermediate are iPSC precursor cells obtained through MET, the intermediate is harvested for three-dimensional suspension culture according to the present invention.

Suspension cultures suitable for the present invention include, but are not limited to, agitating culture, rotating culture, shaking culture, spinning culture, and stirring culture. In the present invention, suspension culture does not include culturing cells in suspension by embedding, encapsulating or attaching the cells in or on a specific solid or semi-solid matrix. Such solid or semi-solid matrix includes, but is not limited to, gels, hydrogels, high polymer scaffolds, and microcarriers. Furthermore, in the present invention, 3D suspension culture does not involve any pretreatment of embedding, encapsulating or attaching cells within or onto a support or matrix prior to suspension culture. Therefore, it can be understood that suspension culture in which cells are embedded in a solid or semi-solid matrix such as a gel or hydrogel (e.g., gel microbeads), or in which cells are seeded or attached on a solid or semi-solid matrix (e.g., microcarriers), is not included within the scope of the 3D suspension culture of the present invention. In the 3D suspension culture of the present invention, cells growing in suspension in a liquid medium mainly rely on cell-cell interactions to gradually form clusters, mass, and/or spheroids. Various suspension culture systems for three-dimensional culture of embryonic stem (ES) cells and iPSCs are known in the art and are also applicable to the 3D reprogramming method of the present invention. Examples of a 3D suspension culture system include, but are not limited to, a roller bottle, an orbital shaker and a fluid system, for example, a roller bottle bioreactor, a rotating bioreactor, a microfluidic bioreactor, and a hollow fiber system. The general principle of bioreactor-based 3D suspension culture systems is to fill a chamber with a cell suspension with an appropriate cell density and provide continuous agitation through stirring, rotation or perfusion of medium via a pump system. Preferably, in the method of the present invention, a stirring or rotating suspension culture system, such as a stirring or rotating suspension bioreactor, for example, a 100 mL- to 1 L-stirring/rotating suspension bioreactor is used. In the 3D suspension culture according to the present invention, by adjusting the stirring or rotating speed, the size of cell mass suspended in the liquid medium can be controlled and maintained; and/or by adjusting fluid shear stress, the pluripotent state of the cells can be modulated and the unwanted differentiation can be suppressed.

In some embodiments, step (c) of the method of the present invention comprises culturing the MET reprogramming intermediate of the present invention in a stirring or rotating suspension culture system, e.g., a roller bottle, an orbital shaker or a bioreactor. In some embodiments, step (c) of the method of the present invention comprises culturing the reprogramming intermediate in a suspension culture at a rotational speed of 30-100 rpm, preferably at approximately 60 rpm, approximately 70 rpm, approximately 80 rpm, or approximately 90 rpm.

In some embodiments, step (c) of the method of the present invention further comprises passaging the spheroid of cells formed after a period of suspension culture for at least one generation, preferably for at least 2 or 3 generations, to obtain a stable cell population of iPSCs. In some embodiments, the passaging of cells is performed when the spheroid of iPSCs reaches a size of 300 to 500 μm. In some embodiments, the passaging of cells is performed once every 4 to 6 days. Preferably, a rho-associated protein kinase (ROCK) inhibitor, preferably Y27632, is added to a medium within the first 12 to 48 h (preferably 24 h) after passaging cells. In some other embodiments, the passaging of cells comprises producing single cells or a cell mass consisting of no more than 200 cells from the spheroid of iPSCs, and inoculating. During passaging, the cells can be dissociated into single cells or small cell mass, for example into a suspension of single cells, by enzymatic digestion or mechanical disaggregation. Any enzyme preparation known in the art that can disaggregate cells into single cells can be used. In an embodiment of the present invention, the enzyme preparation for disaggregation is selected from the group consisting of trypsin/EDTA, TrypLE-Select, collagenase IV and dispase. Preferably, a suspension of single iPSCs is produced from the spheroid of iPSCs by enzymatic digestion (e.g., Accutase enzyme) for passaging cells.

Liquid media for the suspension culture of the present invention are not limited. In some embodiments, the liquid medium according to the present invention is a non-gel liquid medium. Any liquid medium suitable for iPSC culture can be used in the three-dimensional suspension culture according to the present invention. Examples of such medium are known in the art and include, but are not limited to: StemScale PSC Suspension Medium, 2) mTeSRTM3D Media for Human PSC Suspension Culture, 3) Essential 8 medium, 4) StemFit Medium, 5) mTeSRTM3D Media, 6) StemMACS™ iPS-Brew XF, 7) RSeT™ Medium and 8) NutriStem Medium. In some embodiments, the method according to the present invention comprises, in step (c), culturing the cell with StemScale PSC suspension medium.

### Detection of Induced Pluripotent Stem Cells

Pluripotent stem cells (PSCs) are undifferentiated cells that have a short cell cycle and a characteristic gene expression profile, can self-renew and produce any body cell type except extra-embryonic lineages. The quality and/or quantity of the spheroids of 3D-iPSCs can be detected by PSC identification methods known or described in embodiments of the present invention.

Characteristics of pluripotency of cells include, but are not limited to, detectable morphological features, expression of molecular markers and/or biochemical changes associated with pluripotency, and DNA methylation profiles. These hallmarks can be monitored individually or in combination to assess the quality of the 3D-iPSC cells. In some embodiments, the characteristics to be assessed include, but are not limited to: i) round and flat morphology of pluripotent stem cells, ii) expression of pluripotent stem cell markers including SSEA3/4, TRA1-60/81, TRA1-85, TRA2-54, GC™-2, TG343, TG30, CD9, CD29, CD133/prominin, CD140a, CD56, CD73, CD105, CD31, CD34, OCT4, Nanog, Sox2, and any combination thereof, iii) the ability to promote germline transmission in mouse chimeras, iv) teratoma formation, vi) embryoid body formation and trilineage differentiation in vitro, and vii) the reactivation of inactivated X chromosomes.

In some embodiments, the morphology of 3D-iPSC spheroids is examined using a microscope, or the 3D-iPSC spheroids are inoculated into a two-dimensional culture plate and their morphology is observed as they grow, so as to assess the quality and/or quantity of the 3D-iPSCs. In some other embodiments, the reprogramming status of iPSC spheroids is assessed by detecting the expression of pluripotency markers and/or transgenes in the iPSC spheroids through immunofluorescence staining and RT-PCR targeting pluripotency markers and. Preferably, the pluripotency markers are selected from one or more of: OCT4, SOX2, TRA1-60, NANOG and PODXL. Additionally, live-cell staining known in the art can also be used to observe iPSC colonies generated in 3D culture, including, for example, alkaline phosphatase live-cell staining; or live-cell staining using Tra1-60 or Tra1-81 antibodies specifically targeting the iPSC surface marker PODXL. Such antibodies can recognize undifferentiated iPSCs and identify reprogrammed cells among various human cells. In some embodiments, the ability of trilineage differentiation of the obtained 3D-iPSC spheroids is assessed using methods known or described in the embodiments of the present invention.

The human 3D-iPS cells obtained by the 3D reprogramming method of the present invention have the potential for unlimited proliferation in vitro. In some embodiments, the human 3D-iPS cells of the present invention can exhibit a morphology similar to that of embryonic stem cells in a two-dimensional culture in vitro. Specifically, these cells are round with a large nucleus and relatively small cytoplasm volume, and they grow into tightly packed and flat colonies in a manner similar to ES cells. In some embodiments, the human 3D-iPS cells of the present invention express various pluripotency markers, which are selected from, for example, Nanog protein, SSEA family proteins, and TRA family proteins. In some embodiments, the human 3D-iPS cells of the present invention have the potential to differentiate into cells or tissues of three germ layers. In some embodiments, the human 3D-iPS cells of the present invention can cause teratomas formation after being injected into an immunodeficient animal individual.

In some preferred embodiments, the 3D-iPSCs of the present invention form flat and round colonies, express a combination of OCT4, SOX2, NANOG and PODXL, and possess trilineage differentiation potential in vitro.

### Use of the Method of the Present Invention

The use of iPSCs avoids most of the ethical and practical issues resulted from the large-scale clinical application of ES cells. Moreover, patients who receive autologous iPSC-derived grafts may not require lifelong immunosuppressive therapy to prevent transplant rejection. Therefore, iPSCs have broad application prospects in the field of regenerative medicine.

In another aspect of the present invention, provided is a method for producing a downstream cell-derived product. In some embodiments, the method comprises obtaining 3D-iPSCs by the 3D reprogramming method of the present invention, and producing the downstream cell-derived product using the 3D-iPSCs.

The downstream cell-derived product according to the present invention can be used for research and development of stem cells, including, but not limited to, prophylactic or therapeutic applications. For example, in some embodiments, the cells of the present invention (3D-iPSC cells or directed differentiated cells induced therefrom) are introduced into a subject in need thereof, including, but not limited to, a subject in need of regenerating an organ, tissue, or cell type. In some embodiments, the cells are genetically modified prior to their introduction into the subject.

In some embodiments, the pluripotent cells generated according to the method of the present invention are subsequently induced to generate downstream cell-derived products selected from, for example: hematopoietic (stem/progenitor) cells, neural (stem/progenitor) cells (and optionally further differentiated cells such as subtype-specific neurons and oligodendrocytes), pancreatic cells (such as endocrine progenitor cells or pancreatic hormone-expressing cells), hepatocytes, cardiovascular (stem/progenitor) cells (such as cardiomyocytes, endothelial cells, smooth muscle cells), and retinal cells.

Various methods of inducing pluripotent stem cells to differentiate into desired cell types are available. A non-limiting list of recent patent publications describing methods for inducing stem cells differentiation into different cell fates is as follows: U.S. Patent Publication Nos. 2007/0281355, 2007/0269412, 2007/0264709, 2007/0259423, 2007/0254359, 2007/0196919, 2007/0172946, 2007/0141703 and 2007/0134215.

Many diseases can be ameliorated by introducing the pluripotent cells or directionally differentiated cells obtained by the method of the present invention, optionally targeting a specific injured tissue. Examples of the disease include, but are not limited to: neurodegenerative diseases, cerebral infarction, myocardial infarction, heart failure, chronic obstructive pulmonary disease, emphysema, interstitial lung disease and renal insufficiency.

### Examples

### Materials and Methods

### Materials

StemScale PSC Suspension Medium (ThermoFisher, #A4965001)
Y27632 (Tocris, #1254)
Essential 8™ Medium (ThermoFisher, #A1517001)
Penicillin-Streptomycin (P/S) (ThermoFisher, #15140122)
GlutaMAX Supplement (ThermoFisher, #35050061)
L-glutamine (ThermoFisher, #25030081)
B-mercaptoethanol (ThermoFisher, #21985023)
IL-3 (Miltenyi Biotec, #130-095-070)
IL-6 (Miltenyi Biotec, #130-093-932)
SCF (Miltenyi Biotec, #130-096-695)
FLT3L (Miltenyi Biotec, #130-096-477)
DPBS (ThermoFisher, #14190-144)
Accutase (Stem Cell Technologies, #07922)
StemPro™-34 SFM (1X) (ThermoFisher, #10639011)
Dulbecco's Modified Eagle Medium (DMEM) (ThermoFisher, #11960044)
Medium 106 (ThermoFisher, #M106500)
Vitronectin (VTN-N) (ThermoFisher, #A14700)
Fetal bovine serum (FBS) (HyClone, #SH30071.03)
Sodium pyruvate (ThermoFisher, #11360070)
MEM non-essential amino acid solution (NEAA) (NEAA) (ThermoFisher, #11140050)
Trypsin/EDTA solution (TE) (ThermoFisher, #R001100)
UltraPure 0.5M EDTA, pH 8.0 (ThermoFisher, #15575020)
CytoTune 2.0 Sendai Reprogramming Kit (ThermoFisher, #A16517)

### Methods

Human peripheral blood mononuclear cells (PBMCs) and fibroblasts were cultured for reprogramming using CytoTune™ 2.0 Sendai Reprogramming Kit according to the manufacturer's instructions and previously published studies17-19. Briefly, approximately 2-3 days before transfection of reprogramming, human PBMCs or fibroblasts were seeded into wells of a multi-well plate and cultured to ensure complete cell attachment and spreading. On day 0 of reprogramming (i.e., the day of transfection), the human PBMCs and fibroblasts were transduced with CytoTune™ 2.0 Sendai vectors KOS, hc-Myc and hKlf4 at a multiplicity of infection ratio of 5:5:3 or 5:5:6, respectively. The transduced human PBMCs and fibroblasts were cultured according to the manufacturer's protocol until day 18 to day 21 of reprogramming19. Afterwards, the culture plate was treated with enzymes or EDTA to harvest all the cells, which were prepared into a suspension of single cells. The 3D reprogramming of the present invention and the conventional 2D reprogramming as control were separately carried out.

### 3D Reprogramming Procedure

### Establishment of 3D-iPSCs:

In the 3D reprogramming procedure of the present invention, the reprogramming intermediates obtained through mesenchymal-to-epithelial transition (MET) between days 13 to 21 of reprogramming can be harvested and directly transferred to a liquid medium for 3D culture to stabilize the intermediate and generate iPSC spheroids.

Specifically, the reprogramming intermediates harvested on day 21 were cultured in a complete StemScale PSC suspension culture medium in a rotating suspension culture system. To establish 3D-iPSC spheroids, according to the following manner of passaging: the cultures were passaged once every 4-6 days at a ratio of 1:1. By the end of approximately the third passage, stably proliferative 3D-iPSC spheroids with sizes ranging from 300 to 500 μm gradually appeared in the culture and were maintained as described below.

### Maintenance of 3D-iPSC Spheroids:

3D-iPSC spheroids were cultured in a complete StemScale PSC suspension culture medium in an orbital shaker culture system at 70 rpm for maintenance. Cells were passaged once every 4-6 days with Accutase, in which cells were inoculated at a density of 1×10⁵ cells/ml, and the medium was supplemented with 10 μM Y27632 within the first 24 hours after passaging cells. The medium was changed every day by replacing 50% of medium with 50% of fresh complete StemScale PSC suspension culture medium.

### 2D Reprogramming Procedure (as Control)

### Establishment of 2D-iPSC

According to the conventional 2D reprogramming procedures, the reprogramming intermediate was harvested between days 18 to 21 of reprogramming and subjected to 2D-culture on Vitronectin (ThermoFisher) in Essential 8 medium to establish 2D-iPSC cultures (i.e., PO (passage 0) 2D-iPSC cultures). According to the methods of routine maintenance of 2D-iPSCs, the cultures were passaged once every 4 days and the stable 2D-iPSC cultures were usually established at approximately passage 3 (P3).

### Routine Maintenance of 2D-iPSCs

Cells were passaged once every 4 days at a ratio of 1:10-1:20 using 0.5 mM EDTA (ThermoFisher) in plate coated with 5 μg/ml vitronectin (ThermoFisher). During this process, the medium was changed daily, and on the first day of passaging, the medium was supplemented with 10 μM Y-27632 (ROCK inhibitor).

### Results

### Morphological Characterization of Reprogramming Intermediate

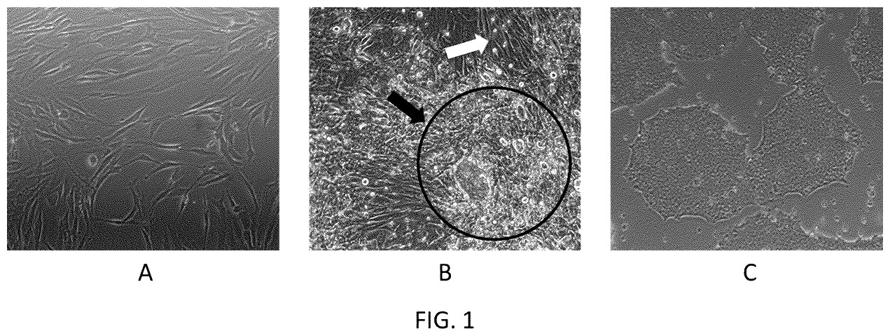

On day 0, day 7, and day 35 of reprogramming, morphological changes of the cells under reprogramming in 2D culture were monitored with a phase contrast microscope to observe the morphology of reprogramming intermediates. The results are shown in FIG. 1.

As shown in FIG. 1, the cell morphology was significantly changed during the reprogramming process.

The left panel (A) shows that the cells exhibited the typical morphology of fibroblasts (somatic cells) on day 0 of reprogramming, and presented in an elongated spindle shape.

The middle panel (B) shows the cells on day 7 of reprogramming. White arrows point to unreprogrammed or reprogramming-resistant cells that still maintained a fibroblast-like morphology. Black arrows point to regions of reprogramming intermediates (within the black-circled area), where the cell colonies lost the fibroblast-like morphology and were dome-shaped, with bright and round cells tightly packed together.

The right panel (C) shows the stable iPSC cultures established through passaging. As can be seen from the image, the morphology of stable iPSC cultures differed from that of reprogramming intermediates in that the cell colonies of the former were flatter with smaller-sized cells, and the cells in the colonies still maintained tightly packed.

Characterization of iPSC Spheroid Obtained by 3D Reprogramming

The morphology of iPSC spheroids obtained by 3D reprogramming was examined using a phase contrast microscope and cell counting was performed using an automated cell counting method based on trypan blue. The expression of multiple pluripotency markers in iPSC spheroids obtained by 3D reprogramming was assessed by immunostaining and qRT-PCR detection. The trilineage differentiation ability of iPSC spheroids obtained by 3D reprogramming was assessed using the STEMdiff™ trilineage differentiation kit, and the differentiation efficiency into endoderm, mesoderm and ectoderm was assessed by flow cytometry.

Morphological Evaluation and Cell Counting of iPSC Spheroid Obtained by 3D Reprogramming

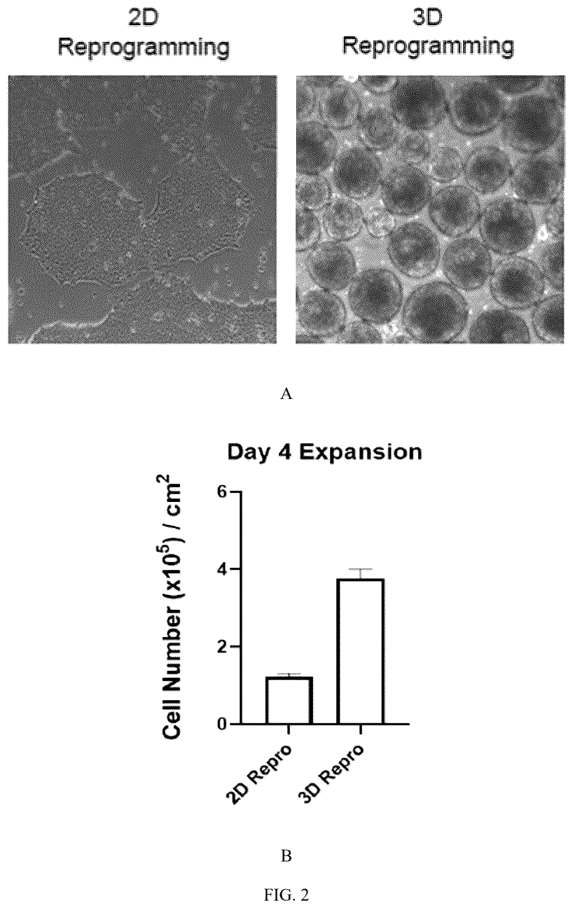

The morphological characterization results of iPSC spheroids obtained by 3D reprogramming are shown in FIG. 2A. FIG. 2A shows the representative images of iPSCs generated by the 2D and 3D reprogramming methods.

After the iPSCs generated by the 2D and 3D reprogramming methods were passaged 10 times, respectively, equal numbers of iPSCs were taken and cultured under either adherent or suspension conditions in 6-well plates for 4 days, respectively. The cells were digested with Accutase into a suspension of single iPS cells, and the cell counting was performed by an automated cell counting method based on trypan blue. FIG. 2B shows the cell yields after 4 days of proliferation of iPSCs obtained by the 2D and 3D reprogramming methods. Compared to the 2D reprogramming method, iPSCs generated and maintained by the 3D reprogramming method of the present invention had an approximately two-fold increase in the iPSC yield within a short period of suspension culture.

### Immunostaining

The immunostaining of 2D-iPSCs and 3D-iPSCs was performed as follows. Cells were fixed in 4% paraformaldehyde (PFA, Sigma) at room temperature for 30 minutes, and then permeabilized with 0.5% Triton X-100 (Sigma) in DPBS (ThermoFisher) for 30 minutes at room temperature. Then, cells were blocked with the blocking buffer (3% bovine serum albumin (BSA, Sigma)+0.1% Tween-20 (Sigma) in Dulbecco's phosphate buffered saline (DPBS, ThermoFisher)) at room temperature for 30 minutes. All antibodies used for immunostaining were prepared in this blocking buffer. Cells were incubated with the primary antibody on a shaker at 4° C. overnight and with the secondary antibody (diluted with blocking buffer at 1:500) at room temperature for 1-2 hours. After the antibodies were labeled, cells were stained with 4′,6-diamidino-2-phenylindole dihydrochloride (DAPI, ThermoFisher) in blocking buffer at a concentration of 5 g/mL for 10 minutes. Subsequently, cells were imaged under an inverted microscope or an inverted confocal microscope (Leica).

Primary antibodies used in this experiment included 1) mouse anti-OCT3/4 IgG2b (Santa Cruz Biotechnology Cat #sc-5279), 2) rat anti-SOX2 IgG2a (Invitrogen Cat #14-9811-82), and 3) mouse anti-TRA-1-60 IgM (BD Biosciences Cat #560071).

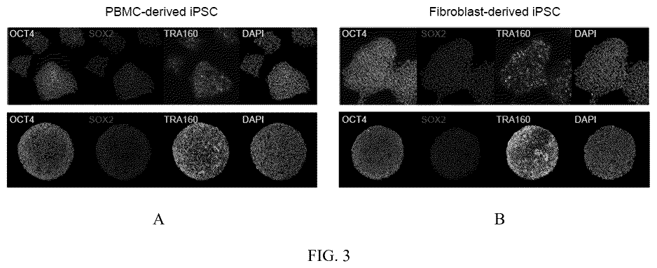

The results are shown in FIG. 3. FIG. 3 shows representative images of iPSCs obtained by 2D and 3D reprogramming methods; wherein pluripotency markers OCT4, SOX2, and TRA-160 were subjected to immunofluorescence staining. Peripheral blood mononuclear cells (PBMC) (A) and fibroblasts (B) were used as the starting somatic cells for 3D-reprograming to generate iPSCs. The iPSCs obtained by the 3D reprogramming method (lower panel) showed a compact spherical morphology and also expressed pluripotency markers OCT4, SOX2 and TRA-160 similar to iPSCs obtained by the 2D reprogramming method (upper panel).

### RT-qPCR Detection

RNA was extracted using the RNeasy micro kit (Qiagen, #74004) and QIAcube kit (Qiagen) according to the manufacturer's instructions. Reverse transcription was performed using the QuantiTect reverse transcription kit (Qiagen, #205311). Real-time PCR was performed using the QuantiFast SYBR Green PCR kit (Qiagen, #204057) in a real-time PCR detection system, with 2 duplicates for each sample. The GAPDH housekeeping gene was used as controls for calculating the relative expression of each pluripotency marker and the transgenes in Sendai virus. The primers used in the RT-qPCR detection were as follows:

Primers for RT-qPCR detection

| Gene | Primers | Sequence |
|---|---|---|
| Control: | | |
| housekeeping | | |
| gene | | |
| GAPDH | Forward primer | CTGGGCTACACTGAGCACC |
| | Reverse primer | AAGTGGTCGTTGAGGGCAATG |
| Gene for | | |
| pluripotency | | |
| marker | | |
| OCT4 | Forward primer | CTGGGTTGATCCTCGGACCT |
| | Reverse primer | CCATCGGAGTTGCTCTCCA |
| SOX2 | Forward primer | GCCGAGTGGAAACTTTTGTCG |
| | Reverse primer | GGCAGCGTGTACTTATCCTTCT |
| NANOG | Forward primer | AAGGTCCCGGTCAAGAAACAG |
| | Reverse primer | CTTCTGCGTCACACCATTGC |
| TRA160 | Forward primer | TCCCAGAATGCAACCCAGAC |
| | Reverse primer | GGTGAGTCACTGGATACACCAA |
| Transgene in | | |
| Sev | | |
| SeV | Forward primer | GGA TCA CTA GGT GAT ATC |
| | | GAG C |
| | Reverse primer | ACC AGA CAA GAG TTT AAG |
| | | AGA TAT GTA TC |
| KOS | Forward primer | ATG CAC CGC TAC GAC GTG |
| | | AGC GC |
| | Reverse primer | ACC TTG ACA ATC CTG ATG |
| | | TGG |
| KLF4 | Forward primer | TTC CTG CAT GCC AGA GGA |
| | | GCC C |
| | Reverse primer | AAT GTA TCG AAG GTG CTC |
| | | AA |
| c-MYC | Forward primer | TAA CTG ACT AGC AGG CTT |
| | | GTC G |
| | Reverse primer | TCC ACA TAC AGT CCT GGA |
| | | TGA TGA TG |

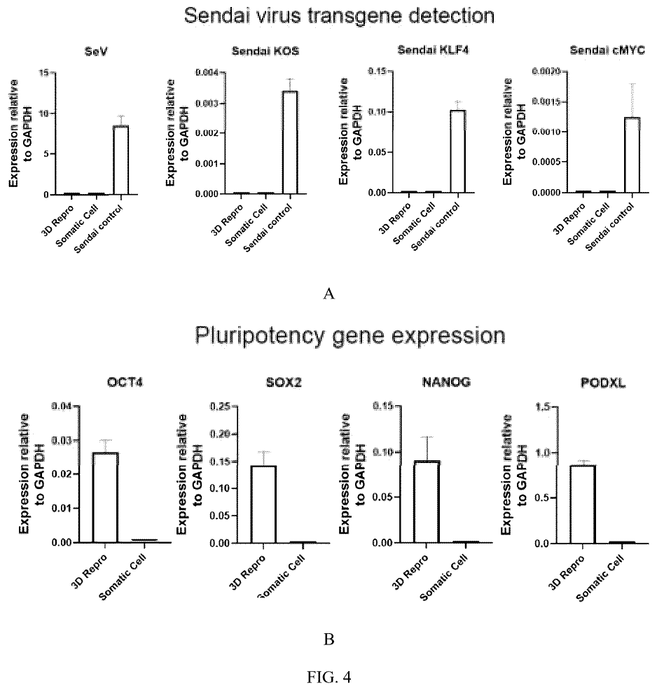

The results are shown in FIG. 4. FIG. 4A shows the results of qRT-PCR detection of transgenes in Sendai virus. Neither transgenes in Sendai virus nor exogenous reprogramming factors were detected in the 3D-reprogrammed iPSCs. FIG. 4B shows the expression of pluripotency markers as detected by qRT-PCR. Compared to the corresponding somatic cell controls, 3D-reprogrammed iPSCs had a higher expression level of pluripotency markers of OCT4, SOX2, NANOG and PODXL.

### Trilineage Differentiation Assay

The trilineage differentiation potential of 2D-reprogramed iPSCs and 3D-reprogramed iPSCs was assessed using the STEMdiff trilineage differentiation kit (Stem Cell Technologies, #05230) according to the manufacturer's instructions. Differentiated cells were analyzed by flow cytometry. Cells were harvested with TrypLE Express, fixed and permeabilized using the Foxp3/transcription factor staining buffer kit (ThermoFisher, #00-5523-00) according to the manufacturer's instructions. The cells were then stained using a two-step antibody labeling procedure, in which the cells were stained in ice for 30 minutes at each labeling step and washed with DPBS between the two steps of labeling. The cells were analyzed using a BD LSR Fortessa Analyzer (BD Biosciences).

Antibodies used in the flow cytomety described above included:
1) rat anti-Nestin (Abcam, Cat #ab6142), 2) mouse anti-PAX6 IgG1 (DHSB, Cat #Pax6), 3) anti-SOX17 antibody (AbCam, Cat #224637), 4) rat anti-FOXA2 (Abcam, Cat #ab256493), 5) mouse anti-CXCR4 IgG2a (PE Miltenyi Biotec, Cat #130-117-690), and 6) Brachyrury monoclonal antibody (Cell signaling technology, Cat #81694s).

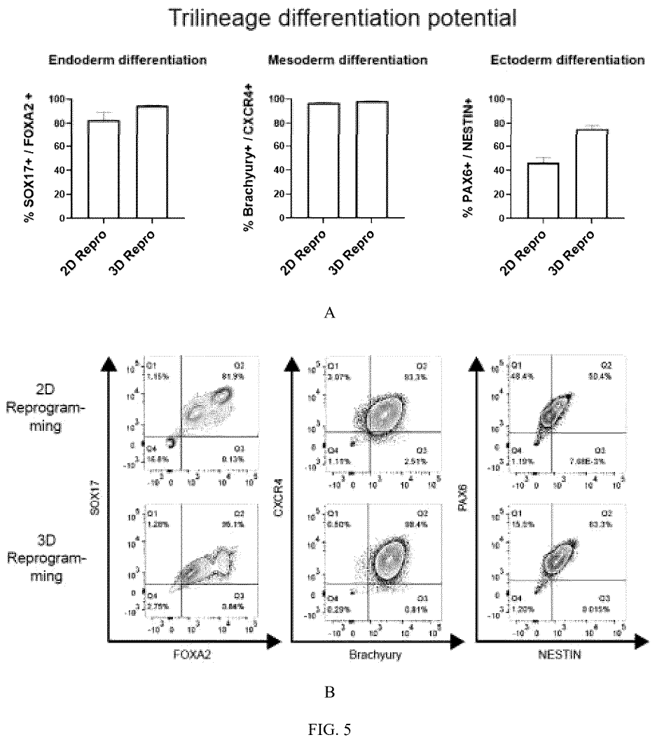

FIG. 5 shows the trilineage differentiation potential of 3D-reprogrammed iPSCs; wherein A shows that compared to 2D-reprogrammed iPSCs, the 3D-reprogrammed iPSCs differentiated into endoderm (SOX17+/FOXA2+) and ectoderm (PAX6+/NESTIN+) with a higher efficiency, and differentiated into mesoderm (Brachyury+/CXCR4+) with a comparable efficiency, and B shows the representative FACS analysis of endoderm, mesoderm, and ectoderm differentiated from 2D-reprogrammed and 3D-reprogrammed iPSCs.

### REFERENCE

1. Thomson, J. A. et al. Embryonic stem cell lines derived from human blastocysts. Science 282, 1145-1147 (1998).
2. Takahashi, K. et al. Induction of pluripotent stem cells from adult human fibroblasts by defined factors. Cell 131, 861-872 (2007).
3. Yamanaka, S. Pluripotent stem cell-based cell therapy-promise and challenges. Cell Stem Cell 27, 523-531 (2020).
4. Omole, A. E. & Fakoya, A. O. J. Ten years of progress and promise of induced pluripotent stem cells: historical origins, characteristics, mechanisms, limitations, and potential applications. PeerJ 6, e4370 (2018).
5. Warren, L. et al. Highly efficient reprogramming to pluripotency and directed differentiation of human cells with synthetic modified mRNA. Cell Stem Cell 7, 618-630 (2010).
6. Carey, B. W. et al. Reprogramming of murine and human somatic cells using a single polycistronic vector. Proc. Natl. Acad. Sci. U.S.A 106, 157-162 (2009).
7. Kim, D. et al. Generation of human induced pluripotent stem cells by direct delivery of reprogramming proteins. Cell Stem Cell 4, 472-476 (2009).
8. Zhou, H. et al. Generation of induced pluripotent stem cells using recombinant proteins. Cell Stem Cell 4, 381-384 (2009).
9. Okita, K. et al. A more efficient method to generate integration-free human iPS cells. Nat. Methods 8, 409-412 (2011).
10. Yu, J. et al. Induced pluripotent stem cell lines derived from human somatic cells. Science 318, 1917-1920 (2007).
11. Caiazzo, M. et al. Defined three-dimensional microenvironments boost induction of pluripotency. Nat. Mater. 15, 344-352 (2016).
12. Caiazzo, M., Tabata, Y. & Lutolf, M. P. Generation of induced pluripotent stem cells in defined three-dimensional hydrogels. Methods Mol. Biol. 1612, 65-78 (2017).
13. Borys, B. S. et al. Optimized serial expansion of human induced pluripotent stem cells using low-density inoculation to generate clinically relevant quantities in vertical-wheel bioreactors. Stem Cells Transl. Med. 9, 1036-1052 (2020).
14. Nogueira, D. E. S. et al. Strategies for the expansion of human induced pluripotent stem cells as aggregates in single-use Vertical-Wheel™ bioreactors. J. Biol. Eng. 13, 74 (2019).
15. Lei, Y. & Schaffer, D. V. A fully defined and scalable 3D culture system for human pluripotent stem cell expansion and differentiation. Proc. Natl. Acad. Sci. U.S.A 110, E5039-48 (2013).
16. Rohani, L. et al. Stirred suspension bioreactors maintain naïve pluripotency of human pluripotent stem cells. Commun. Biol. 3, 492 (2020).
17. Liu, X. et al. Comprehensive characterization of distinct states of human naive pluripotency generated by reprogramming. Nat. Methods 14, 1055-1062 (2017).
18. Tong, J. et al. Generation of four iPSC lines from peripheral blood mononuclear cells (PBMCs) of an attention deficit hyperactivity disorder (ADHD) individual and a healthy sibling in an Australia-Caucasian family. Stem Cell Res. 34, 101353 (2019).
19. CytoTune™-iPS 2.0 Sendai Reprogramming Kit. https://www.thermofisher.com/order/catalog/product/A16517.

1. A method for producing an induced pluripotent stem cell (iPSC) by reprogramming a somatic cell, comprising:
(a) contacting the somatic cell with a reprogramming factor to perform reprogramming treatment on the somatic cell,
(b) culturing the somatic cell treated in step (a) under a 2D culture condition for a period sufficient to obtain a reprogramming intermediate, wherein the reprogramming intermediate is characterized by at least 20%, 30%, 40%, or 50% of cells being iPSC precursor cells obtained through mesenchymal-to-epithelial transition (MET), and
(c) harvesting the reprogramming intermediate obtained in step (b), and culturing the reprogramming intermediate in a liquid medium under a 3D suspension culture condition to generate a spheroid of reprogrammed iPSCs,
wherein the somatic cell is a non-human primate or human somatic cell.

2. The method according to claim 1, wherein the iPSC precursor cells obtained through MET lose original morphological characteristic of the somatic cell and are in a MET morphology characterized by domed, tightly packed cell colonies and optionally an increased nuclear-to-cytoplasmic ratio.

3. The method according to claim 1, wherein the iPSC precursor cells obtained through MET express a pluripotency marker of TRA-1-60 and/or SSEA3.

4. The method according to claim 1, wherein step (c) comprises harvesting the reprogramming intermediate at approximately day 7 to day 21 of reprogramming, more preferably at approximately day 10 to day 18 of reprogramming, for example, harvesting the reprogramming intermediate on day 10, day 11, day 12, day 13, day 14, day 15, day 16, day 17, or day 18.

5. The method according to claim 1, wherein the somatic cell is a somatic cell or cell line from an adult individual.

6. The method according to claim 1, wherein the somatic cell is a human blood cell, for example, a human peripheral blood mononuclear cell (PBMC).

7. The method according to claim 1, wherein the somatic cell is a human fibroblast.

8. The method according to claim 1, wherein in step (c), the 3D suspension culture is a suspension culture system with stirring or rotating, for example, a roller bottle, an orbital shaker or a bioreactor.

9. The method according to claim 1, wherein step (c) further comprises passaging the spheroid of cell obtained after a period of suspension culture for at least one generation, preferably for at least 2 or 3 generations, to obtain a stable cell population of iPSCs.

10. The method according to claim 1, wherein the reprogramming factor is a reprogramming transcription factor, and step (a) comprises introducing one or more reprogramming factors or a nucleic acid encoding the reprogramming factor into the somatic cell to increase expression or protein amount of the reprogramming factor in the somatic cell.

11. The method according to claim 1, wherein step (a) comprises delivering the reprogramming factor to the somatic cell using an integrating virus, a non-integrating virus, an mRNA, an episomal plasmid, or a protein;
preferably, step (a) comprises delivering the reprogramming factor to the somatic cell using a non-integrating virus, in particular a Sendai virus vector.

12. The method according to claim 1, wherein the one or more reprogramming factors comprise or consist of at least three or at least four of Klf4, c-Myc, Oct3/4, Sox2, Nanog and Lin28;
preferably, the one or more reprogramming factors comprise or consist of Oct3/4, Sox2, c-Myc and Klf4.

13. The method according to claim 1, wherein step (c) comprises culturing the reprogramming intermediate in suspension at a rotating speed of 30 to 100 rpm, preferably at a rotating speed of approximately 70 rpm.

14. The method according to claim 1, wherein in step (c), the 3D suspension culture is carried out in an iPSC maintenance medium, preferably in StemScale PSC suspension medium.

15. A method for maintaining or expanding 3D-reprogrammed iPSCs, comprising:
(i) obtaining the spheroid of iPSC reprogrammed from the somatic cell according to the method of claim 1; and
(ii) maintaining or expanding the iPSC under a 3D suspension culture condition.

16. The method according to claim 15, wherein the method further comprises a step of passaging cells after step (i) and before step (ii).

17. The method according to claim 15, wherein the passaging cells is performed when the spheroid of iPSCs reaches a size of 300 to 500 μm.

18. The method according to claim 15, wherein the maintaining iPSCs comprises passaging cells once every 4 to 6 days;
preferably, adding a rho-associated protein kinase (ROCK) inhibitor, preferably Y27632, to a medium within the first 12 to 48 h (preferably 24 h) after passaging cells.

19. The method according to claim 15, wherein the passaging cells comprises producing single cells or a cell mass consisting of no more than 200 cells from the spheroid of iPSCs, and inoculating;
preferably, producing a suspension of single cells from the spheroid of iPSCs by enzymatic digestion (for example Accutase enzyme) for passaging cells.

20. A method for producing a downstream cell-derived product, comprising obtaining iPSCs by the method according to claim 1, and producing the downstream cell-derived product by using the iPSCs.
